(19) United States
(12) Patent Application Publication
(10) Pub. No.: US 2025/0345037 A1
(43) Pub. Date: Nov. 13, 2025

(54) IMPROVED FOUR-DIMENSIONAL (4-D) ULTRASOUND IMAGING

(71) Applicant: GE Precision Healthcare LLC, Waukesha, WI (US)

(72) Inventors: Svetoslav Ivanov Nikolov, Søborg (DK); Fredrik Gran, Limhamn (DK); Lars Nordahl Moesner, Glostrup (DK)

(73) Assignee: GE Precision Healthcare LLC, Waukesha, WI (US)

(21) Appl. No.: 18/661,836

(22) Filed: May 13, 2024

(51) Int. Cl. A61B 8/00; A61B 8/14; G01S 7/52; G01S 15/89

(52) U.S. Cl. CPC A61B 8/463; A61B 8/145; A61B 8/4488; G01S 7/52095; G01S 15/8925; G01S 15/8993

(57) ABSTRACT

An ultrasound imaging system includes a transmit circuit configured to generate ultrasound element excitation pluses based on an acquisition mode that includes at least two different types of ultrasound acquisitions for a single ultrasound imaging scan, a transducer array with elements configured to transmit ultrasound pressure waves based on the at least two different types of ultrasound acquisitions, and receive, for each of the at least two different types of ultrasound acquisitions, a corresponding echo signal, a beamformer configured to independently beamform the echo signals for the at least two different types of ultrasound acquisitions, an image generator configured to generate an image for each of the at least two different types of ultrasound acquisitions based on the corresponding echo signal, and a display configured to concurrently display the images for each of the at least two different types of ultrasound acquisitions.

### FIELD

The following generally relates to ultrasound imaging, and more particularly to improved four-dimensional (4-D) ultrasound imaging.

### BACKGROUND

Ultrasound imaging provides real-time imaging of information about the interior of an object or a subject such as tissue, organs, etc. An example ultrasound imaging system includes an ultrasound imaging probe and a console. The ultrasound imaging probe includes a transducer array. The transducer array is configured to transmit a pressure wave and receive echoes produced in response to the pressure wave interacting with structure such as tissue, blood cells, etc. The transducer array converts the echoes into analog signals. The console includes signal processing hardware and software, a user interface and a display monitor. The console processing the analog signals to produce images for B-mode, C-mode, A-mode, Doppler, color-flow, elastography, three-dimensional (3-D), four-dimensional (4-D), etc. applications.

Two-dimensional (2-D) transducer arrays allow imaging a combination of planes (e.g., B-mode imaging) and slices (e.g., C-mode imaging). As utilized herein, a plane originates from a transducing surface of the transducer array, and a slice does not originate from the transducing surface of the transducer array and extends across multiple planes. FIGS. 1, 2, 3, 4, 5, 6 and 7 collectively schematically illustrates planes and slices. FIG. 1 schematically illustrates a perspective view of a plane 102 in connection with a transducing surface 104 of a 2-D transducer array 106. The plane 102 originates at the transducing surface 104, is orthogonal to the transducing surface 104, and extends in one of two directions (azimuth or elevation) of the 2-D transducer array 106. In another example, the plane 102 originates at the transducing surface 104, but is not orthogonal to the transducing surface 104.

FIG. 2 schematically illustrates a perspective view of a plane 202 in connection with the transducing surface 104 of the 2-D transducer array 106. Similar to the plane 102 in FIG. 1, the plane 202 originates at the transducing surface 104 of the 2-D transducer array 106 and is orthogonal to the transducing surface 104 of the 2-D transducer array 106. However, the plane 202 extends in the other of the two directions (elevation or azimuth) of the 2-D transducer array 106 and is orthogonal to the plane 102 in FIG. 1. In another example, the plane 202 originates at the transducing surface 104, but is not orthogonal to the transducing surface 104. FIG. 3 schematically illustrates a perspective view of the planes 102 and 202 together in connection with the transducing surface 104 of the 2-D transducer array 106. In another example, the plane 202 also is not orthogonal to the plane 102.

FIG. 4 schematically illustrates a perspective view of multiple slices, including a slice 402, . . . , and a slice 404, in connection with the transducing surface 104 of the 2-D transducer array 106. The slice 402, . . . , and the slice 404 are part of a same volume in front of the transducing surface 104, do not originate at the transducing surface 104, are parallel to the transducing surface 104, and extend across multiple planes that originate at the transducing surface 104, e.g., the plane 102 of FIG. 1 and the plane 202 of FIG. 2. In general, a spatial orientation of a slice with respect to the transducing surface 104 is defined by an angle α in azimuth between a line normal to the transducing surface 104 and the slice, and an angle β in elevation between the line normal to the transducing surface 104 the slice.

In FIG. 4, the slice 402, . . . , and the slice 404 are parallel to the transducing surface 104 and α=0° and β=0°. When α is in range of 0°<α<90°, the slice is tilted in azimuth. When β is in range of 0°<β<90°, the slice is tilted in elevation. When α and β are concurrently in range of 0°<α<90°, the slice is tilted in both azimuth and elevation. Where one of α or β=90°, the slice becomes a plane that originates at the transducing surface 104 of the 2-D transducer array 106, similar to the plane 102 of FIG. 1 and the plane 202 of FIG. 2. An example of a slice 502 tilted in azimuth (0°<α<90°) but not in elevation is schematically illustrated in FIG. 5, which schematically illustrates a perspective view of the tilted slice 502 in connection with the transducing surface 102 of the 2-D transducer array 106. The slice 502 is part of the same volume as the slice 402, . . . , and the slice 404 of FIG. 4.

FIG. 6 schematically illustrates a perspective view of the plane 102, the plane 202, and the slice 402 in connection with the transducing surface 104 of the 2-D transducer array 106. In FIG. 6, the plane 102 and the plane 202 are orthogonal to the transducing surface 104, the slice 402 is parallel to the transducing surface 104, and the plane 102, the plane 202 and the slice 402 are orthogonal to each other. FIG. 7 schematically illustrates a side view of FIG. 6 along one of the two directions (azimuth or elevation) in connection with the transducing surface 104 of the 2-D transducer array 106. With reference to FIGS. 6 and 7, again, the plane 102 and the plane 202 extend from but do not have to be orthogonal to the transducing surface 104, and the slice 402 extends across the plane 102 and the plane 202, but does not have to be parallel to the transducing surface 104.

Two-dimensional (2-D) transducer arrays are capable of acquiring data for generating planes and slices in connection with 4-D imaging, e.g., 4-D cardiac imaging. With a 2-D transducer array, the user can electronically steer and focus the ultrasound beam anywhere in front of transducer array. A higher frame rate is achieved by utilizing a wider acquisition beam, which utilizes fewer emissions per frame relative to a narrower acquisition beam. However, a wider acquisition beam reduces image quality (resolution and/or contrast) relative to a narrower acquisition beam. Higher image quality is achieved by utilizing a narrower acquisition beam, which utilizes a greater number of emissions per frame relative to a wider acquisition beam. However, a narrower acquisition beam reduces the frame rate.

Example workflow, e.g., for 4-D cardiac imaging, etc., has included acquiring a volume of data with a single acquisition scheme and then generating and concurrently displaying one or more B-mode planes and one or more C-mode slices selected by a user. For cardiac imaging approaches that benefit from higher frame rates, image quality has been sacrificed for speed. Techniques such as parallel receive beamforming, etc. have been utilized to improve frame rate, and/or coherent compounding, etc. have been used to increase image quality. Unfortunately, as described above, with existing approaches there is a tradeoff between speed and image quality. In view of at least the foregoing, there is an unresolved need for an improved approach that increases both speed and image quality.

### SUMMARY

Aspects of the application address the above matters, and others. This summary introduces concepts that are described in more detail in the detailed description. It should not be used to identify essential features of the claimed subject matter, nor to limit the scope of the claimed subject matter.

In one aspect, an ultrasound imaging system includes a transmit circuit configured to generate ultrasound element excitation pluses based on an acquisition mode that includes at least two different types of ultrasound acquisitions for a single ultrasound imaging scan. The ultrasound imaging system further includes a transducer array with elements configured to transmit ultrasound pressure waves based on the at least two different types of ultrasound acquisitions. The transducer array is further configured to receive, for each of the at least two different types of ultrasound acquisitions, a corresponding echo signal. The ultrasound imaging system further includes a beamformer configured to independently beamform the echo signals for the at least two different types of ultrasound acquisitions. The ultrasound imaging system further includes an image generator configured to generate an image for each of the at least two different types of ultrasound acquisitions based on the corresponding echo signal. The ultrasound imaging system further includes a display configured to concurrently display the images for each of the at least two different types of ultrasound acquisitions.

In another instance, one of the at least two different types of ultrasound acquisitions includes an image plane acquisition and another of the at least two different types of ultrasound acquisitions includes an image slice acquisition. In another instance, the transducer array interleaves transmissions for the image plane and the image slice. In another instance, the transducer array transmits a first pressure wave for acquiring a sub-set of scanlines for the image plane and a second pressure for acquiring a volume of data for the image slice. In another instance, the transducer array interleaves transmissions for two image planes and the image slice. In another instance, the transducer array transmits a first pressure wave for acquiring a sub-set of scanlines for a first image plane, a second pressure for acquiring a volume of data for the image slice, and a third pressure wave for acquiring a sub-set of scanlines for a second image plane. In another instance, the first image plane is in azimuth and the second image plane is in elevation. In another instance, the transducer array transmits a first pressure wave for acquiring a sub-set of scanlines for a first image plane, a second pressure for acquiring a sub-set of scanlines for a second image plane, and a third pressure wave for acquiring a volume of data for the image slice. In another instance, the first image plane is in azimuth and the second image plane is in elevation. In another instance, the image includes a first image plane, and the first image plane has a first image quality that is greater than a second image quality of a second image generated from a volume of data, given a same frame rate.

In another aspect, a computer-implemented method includes generating ultrasound element excitation pluses based on an acquisition mode that includes at least two different types of ultrasound acquisitions for a single ultrasound imaging scan. The computer-implemented method further includes transmitting ultrasound pressure waves based on the at least two different types of ultrasound acquisitions. The computer-implemented method further includes receiving, for each of the at least two different types of ultrasound acquisitions, a corresponding echo signal. The computer-implemented method further includes beamforming the echo signals for the at least two different types of ultrasound acquisitions. The computer-implemented method further includes generating an image for each of the at least two different types of ultrasound acquisitions based on the corresponding echo signal. The computer-implemented method further includes displaying, concurrently, the images for each of the at least two different types of ultrasound acquisitions.

In another instance, one of the at least two different types of ultrasound acquisitions includes an image plane acquisition and another of the at least two different types of ultrasound acquisitions includes an image slice acquisition, and further comprising interleaving transmissions for the image plane and the image slice. In another instance, the interleaving comprises transmitting a first pressure wave for acquiring a first sub-set of scanlines for the image plane, receiving a first echo signal in response to the first pressure wave, transmitting a second pressure for acquiring a volume of data for the image slice, receiving a second echo signal in response to the second pressure wave, transmitting at least a third pressure wave for acquiring a second sub-set of scanlines for the image plane, receiving at least a third echo signal in response to the third pressure wave, and generating the image plane based on the first and third echo signals and the image slice based on the second echo signal.

In another instance, the interleaving comprises: transmitting a first pressure wave for acquiring a first sub-set of scanlines for a first image plane, receiving a first echo signal in response to the first pressure wave, transmitting a second pressure for acquiring a volume of data for the image slice, receiving a second echo signal in response to the second pressure wave, transmitting a third pressure wave for acquiring a first sub-set of scanlines for a second image plane, receiving a third echo signal in response to the third pressure wave, and generating the first image plane based on the first echo signal, the image slice based on the second echo signal, and the second image plane based on the third echo signal. In another instance, the interleaving comprises transmitting a first pressure wave for acquiring a first sub-set of scanlines for a second image plane, receiving a first echo signal in response to the first pressure wave, transmitting a second pressure wave for acquiring a first sub-set of scanlines for a second image plane, receiving a second echo signal in response to the third pressure wave, transmitting a third pressure wave for acquiring a volume of data for the image slice; receiving a third echo signal in response to the third pressure wave, and generating the first image plane based on the first echo signal, the second image plane based on the second echo signal, and the image slice based on the third echo signal.

In another aspect, a computer readable medium is encoded with computer executable instructions, which, when executed by a processor, cause the processor to generate ultrasound element excitation pluses based on an acquisition mode that includes at least two different types of ultrasound acquisitions for a single ultrasound imaging scan. The instructions further cause the processor to transmit ultrasound pressure waves based on the at least two different types of ultrasound acquisitions. The instructions further cause the processor to receive, for each of the at least two different types of ultrasound acquisitions, a corresponding echo signal. The instructions further cause the processor to beamform the echo signals for the at least two different types of ultrasound acquisitions. The instructions further cause the processor to generate an image for each of the at least two different types of ultrasound acquisitions based on the corresponding echo signal. The instructions further cause the processor to display, concurrently, the images for each of the at least two different types of ultrasound acquisitions.

In another instance, the one of the at least two different types of ultrasound acquisitions includes an image plane acquisition and another of the at least two different types of ultrasound acquisitions includes an image slice acquisition, and the computer executable instructions further cause the processor to interleave transmissions for the image plane and the image slice. In another instance, the one the computer executable instructions further cause the processor to transmit a first pressure wave for acquiring a first sub-set of scanlines for the image plane, receive a first echo signal in response to the first pressure wave, transmit a second pressure for acquiring a volume of data for the image slice, receive a second echo signal in response to the second pressure wave, transmit at least a third pressure wave for acquiring a second sub-set of scanlines for the image plane, receive at least a third echo signal in response to the third pressure wave, and generate the image plane based on the first and third echo signals and the image slice based on the second echo signal.

In another instance, the computer executable instructions further cause the processor to transmit a first pressure wave for acquiring a first sub-set of scanlines for a first image plane, receive a first echo signal in response to the first pressure wave, transmit a second pressure for acquiring a volume of data for the image slice, receive a second echo signal in response to the second pressure wave, transmit a third pressure wave for acquiring a first sub-set of scanlines for a second image plane, receive a third echo signal in response to the third pressure wave, and generate the first image plane based on the first echo signal, the image slice based on the second echo signal, and the second image plane based on the third echo signal. In another instance, the computer executable instructions further cause the processor transmit a first pressure wave for acquiring a first sub-set of scanlines for a second image plane, receive a first echo signal in response to the first pressure wave, transmit a second pressure wave for acquiring a first sub-set of scanlines for a second image plane, receive a second echo signal in response to the third pressure wave, transmit a third pressure wave for acquiring a volume of data for the image slice, receive a third echo signal in response to the third pressure wave, and generate the first image plane based on the first echo signal, the second image plane based on the second echo signal, and the image slice based on the third echo signal.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a perspective view of a first plane originating from a transduce surface of a 2-D transducer array in connection with the transducing surface of the 2-D transducer array, in accordance with an aspect of an embodiment(s) herein.

FIG. 2 schematically illustrates a perspective view of a second different plane originating from the transduce surface of the 2-D transducer array in connection with the transducing surface of the 2-D transducer array, in accordance with an embodiment(s) herein.

FIG. 3 schematically illustrates a perspective view showing the first plane of FIG. 1 and the second different plane of FIG. 2 together in connection with the transducing surface of the 2-D transducer array, in accordance with an embodiment(s) herein.

FIG. 4 schematically illustrates a perspective view of multiple slices parallel to the transducing surface of the 2-D transducer array in connection with the transducing surface of the 2-D transducer array, in accordance with an embodiment(s) herein.

FIG. 5 schematically illustrates a perspective view of a single slice tiled in azimuth or elevation and not originating from the transduce surface of the 2-D transducer array in connection with the transducing surface of the 2-D transducer array, in accordance with an embodiment(s) herein.

FIG. 6 schematically illustrates a perspective view showing the first plane of FIG. 1, the second different plane of FIG. 2, and a slice of FIG. 4 together in connection with the transducing surface of the 2-D transducer array, in accordance with an embodiment(s) herein.

FIG. 7 schematically illustrates a side view showing the first plane of FIG. 1, the second different plane of FIG. 2, and a slice of FIG. 4 together in connection with the transducing surface of the 2-D transducer array, in accordance with an embodiment(s) herein.

FIG. 8 schematically illustrates a non-limiting example of an ultrasound imaging system, in accordance with an aspect of an embodiment(s) herein.

FIG. 9 schematically illustrates an example of acquisition modes of the ultrasound imaging system, in accordance with an embodiment(s) herein.

FIG. 10 schematically illustrates an example of a beamformer of the ultrasound imaging system, in accordance with an embodiment(s) herein.

FIG. 11 schematically illustrates an example of a beamformer of the ultrasound imaging system that includes multiple receive beamformers, in accordance with an embodiment(s) herein.

FIG. 12 schematically illustrates example displays of one or more B-mode planes and C-mode slices, in accordance with an embodiment(s) herein.

FIG. 13 illustrates a non-limiting example of a flow chart for a computer-implemented method for generating a B-mode image plane(s) and a C-mode image slice(s) for a combined B-mode image plane/C-mode image slice scan, in accordance with an embodiment(s) herein.

FIG. 14 illustrates a non-limiting example of a flow chart for a computer-implemented method for generating a B-mode image plane and a C-mode image slice(s) for a combined B-mode image plane/C-mode image slice scan, in accordance with an embodiment(s) herein.

FIG. 15 illustrates a non-limiting example of a flow chart for a computer-implemented method for generating two B-mode image planes and a C-mode image slice(s) for a combined B-mode image plane/C-mode image slice scan, in accordance with an embodiment(s) herein.

FIG. 16 illustrates another non-limiting example of a flow chart for a computer-implemented method for generating two B-mode image planes and a C-mode image slice(s) for a combined B-mode image plane/C-mode image slice scan, in accordance with an embodiment(s) herein.

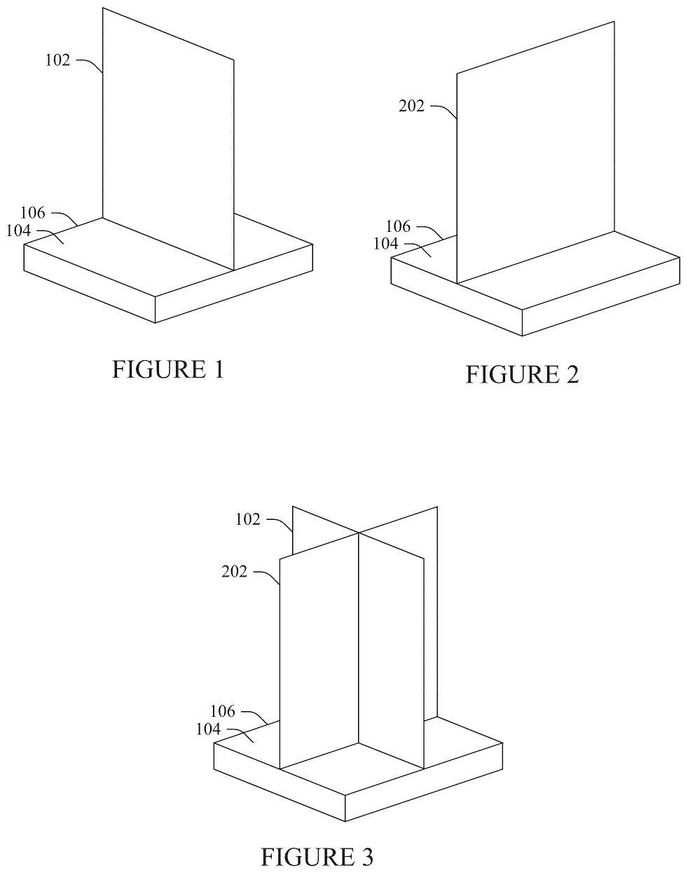

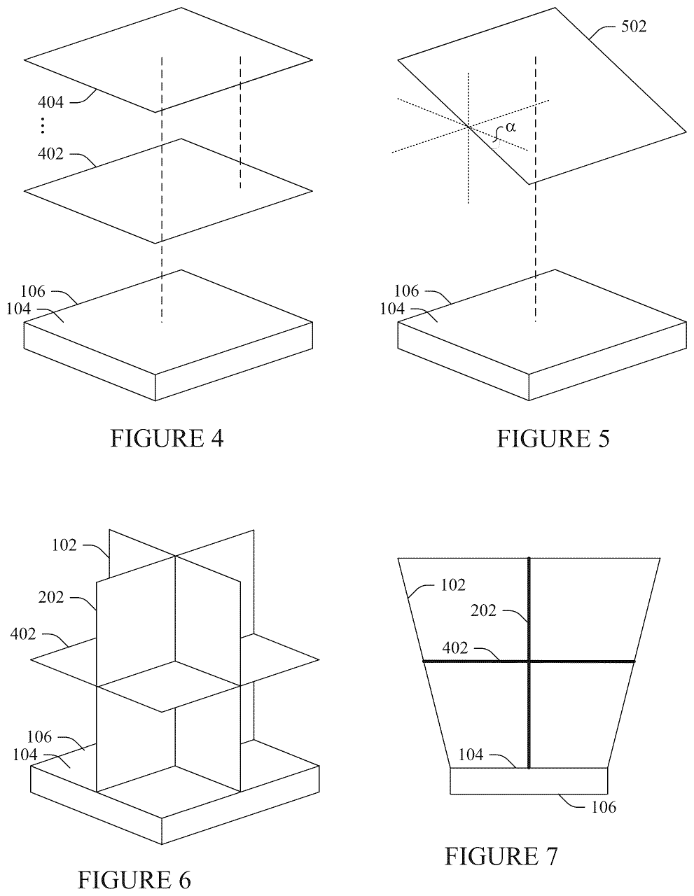

### DETAILED DESCRIPTION

Embodiments of the present disclosure will now be described, by way of example, with reference to the figures, in which an ultrasound imaging system, a method and/or a computer readable medium that combines different types of data acquisitions schemes for acquiring data for B-mode planes and C-mode slices, including focused narrower transmission beams to acquire scanplanes of data for B-mode planes and wider transmission beams to acquire a volume data for C-mode slices. In one instance, the different types of data acquisition schemes are interleaved such that multiple sets of scanlines are acquired for a B-mode plane(s) with volume data for C-mode slices acquired between acquiring the sets of scanlines. In one instance, the approach described herein utilizes a combination of hardware and software beamforming to generate B-mode and C-mode images.

As discussed above, 2-D transducer arrays allow for imaging a combination of planes and slices, e.g., in connection with 4-D imaging such as 4-D cardiac imaging. Example workflow, e.g., for 4-D cardiac imaging, has included acquiring a volume of data with a single acquisition scheme and then generating and concurrently displaying one or more B-mode planes and one or more C-mode slices selected by a user. Unfortunately, with existing approaches there is a tradeoff between speed and image quality, and cardiac scans, which can benefit from higher frame rates, generally have sacrificed image quality for speed. The approach described herein allows for arbitrary 3-D spatial orientation with higher frame rate and higher image quality, relative to configurations not employing the approach described herein. The approach described herein can also be utilized with known approaches for increasing frame rate (e.g., parallel receive beamforming, etc.) and/or increasing image quality (e.g., coherent compounding, synthetic transmit focusing, adaptive focusing, etc.).

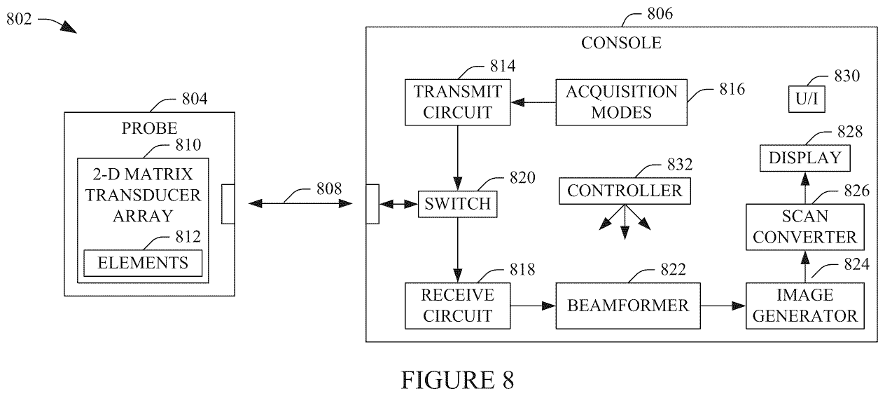

Turning to FIG. 8, a non-limiting example of an ultrasound system 802 is schematically illustrated. The ultrasound system 802 includes an ultrasound imaging probe 804 and a console 806. The ultrasound imaging probe 804 and the console 806 interface with each other over a communication channel 808, which includes a wired communication channel (e.g., respective interfaces, a cable, complimentary electromechanical connectors, etc.) and/or wireless technology (e.g., Wi-Fi, etc.). In another instance, the ultrasound imaging probe 804 and the console 806 are integrated in a same housing such as part of a hand-held ultrasound system, etc.

The ultrasound imaging probe 804 includes a two dimensional (2-D) matrix transducer array 810. The 2-D matrix transducer array 810 includes a 2-D matrix array of transducer elements 812. Examples of suitable 2-D matrix arrays include N×M arrays, where N and M are integers equal to or greater than one. Example of N and M are 32, 48, 64, 96, 128, 192, 256, and/or other number of elements, square, rectangular, circular, and/or otherwise. The 2-D matrix transducer array 810 can be linear, curved, and/or otherwise shaped, fully populated, sparse and/or a combination thereof, etc. The one or more transducer elements 812 are configured to convert an excitation electrical signal to an ultrasound pressure field and convert a reflected ultrasound pressure field to an electrical signal.

By way of non-limiting example, the one or more transducer elements 812 can be selectively excited via an excitation electrical (pulsed) signal, which causes at least a sub-set of the one or more transducer elements 812 to transmit an ultrasound pressure field into an examination or scan field of view for one or more B-mode plane acquisitions and/or a C-mode slice volume acquisition. The ultrasound pressure field may include a focused beam, a defocused beam, a planar wave, and/or other ultrasound pressure field. The one or more transducer elements 812 receive echo signals and generate analog electrical signals indicative thereof. The echo signals are generated in response to the transmitted ultrasound pressure field interacting with structure, such as tissue and/or blood cells flowing in a portion of a vessel.

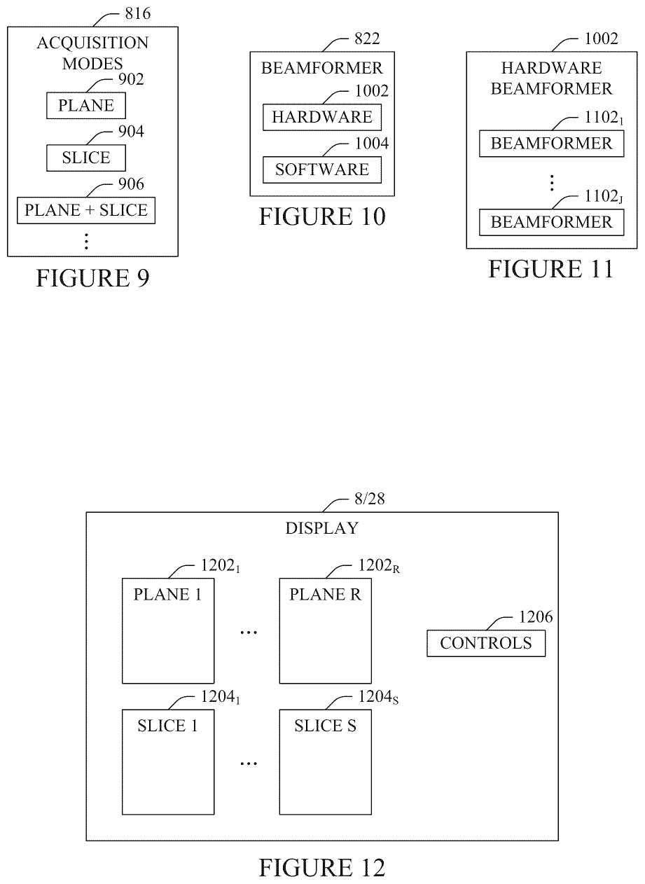

The console 806 includes a transmit circuit 814 configured to generate the excitation electrical signal provided to the 2-D matrix transducer array 810. In this example, the transmit circuit 814 generates the excitation electrical signal based on an acquisition mode of available acquisition modes 816. Briefly turning to FIG. 9, a non-limiting example of the acquisition modes 816 is schematically illustrated. In this example, the acquisition modes 816 include at least a plane acquisition mode 902 (“PLANE”), a slice acquisition mode 904 (“SLICE”), and a plane+slice acquisition mode 906 (“PLANE+SLICE”). In one instance, the plane acquisition mode 902 transmits focused narrower beams, the slice acquisition mode 904 transmits wider beams, and the plane+slice acquisition mode 906 transmits a combination of focused narrower beams and wider beams.

In one instance, the transmit circuit 814 excites the elements 812 to interleave the transmission of focused narrower beams and wider beams to concurrently acquire data for B-mode planes and C-mode slices. In one instance, the interleave pattern is P1, S, P1, S, P1, S, P1, S, P1, S, . . . , where P1 represents a focused narrow beam transmission to acquire scanlines for a B-mode plane originating at transducing surface of the 2-D matrix transducer array 810 and S represents a wide beam transmission to acquire a volume of data for one or more C-mode slices, which do not originate at the transducing surface of the 2-D matrix transducer array 810 and instead extend across planes originating at transducing surface of the 2-D matrix transducer array 810.

The pattern is repeated to acquire data for scanlines for the entire B-mode plane, and then repeated again to refresh the B-mode plane. The set of the focused narrow beam transmissions P1 collectively include scanlines for the entire scanplane. In one instance, each C-mode slice transmission S covers an entire volume being imaged. In another instance, each C-mode slice transmission S covers a different sub-volume (with or without overlap) of the entire volume being imaged, and the sub-volumes from each transmission are combined to cover the entire volume being imaged. The subject interleave pattern acquires data for a single B-mode plane and one or more C-mode slices. In one instance, P1 acquires data to generate an axial image or a sagittal image and S acquires data to generate a coronal image.

For two B-mode planes (e.g., one in azimuth and one in elevation) and one or more C-mode slices, an example interleave pattern includes P1, S, P2, S, P1, S, P2, S, P1, S, . . . , where P1 represents a focused narrow beam transmission to acquire scanlines for a first B-mode plane originating at transducing surface of the 2-D matrix transducer array 810, P2 represents a focused narrow beam transmission to acquire scanlines for a second different B-mode plane originating at transducing surface of the 2-D matrix transducer array 810, and S again represents a wide beam transmission to acquire a volume of data for one or more C-mode slices, which do not originate at the transducing surface of the 2-D matrix transducer array 810 and instead extend across planes originating at transducing surface of the 2-D matrix transducer array 810. In one instance, P1 acquires data to generate an axial image or a sagittal image, P2 acquires data to generate the other of the axial image or the sagittal image, and S acquires data to generate a coronal image.

For two B-mode planes (e.g., one in azimuth and one in elevation) and one or more C-mode slices, another example interleave pattern includes P1, P2, S, P1, P2, S, P1, P2, S, P1, P2, S, . . . , where, again, P1 represents a focused narrow beam transmission to acquire scanlines for a first B-mode plane originating at transducing surface of the 2-D matrix transducer array 810, P2 represents a focused narrow beam transmission to acquire scanlines for a second different B-mode plane originating at transducing surface of the 2-D matrix transducer array 810, and S represents a wide beam transmission to acquire a volume of data for one or more C-mode slices, which do not originate at the transducing surface of the 2-D matrix transducer array 810 and instead extend across planes originating at transducing surface of the 2-D matrix transducer array 810. Again, P1 acquires data to generate an axial image or a sagittal image, P2 acquires data to generate the other of the axial image or the sagittal image, and S acquires data to generate a coronal image.

Other interleaved transmit patterns are also contemplated herein. In general, the frame rate FR for the approached described herein can be determined as shown in EQUATION 1:

$$FR = \frac{PRF}{( S + 1 ) * {N}_{b}} , \qquad EQUATION 1$$

where PRF represents the pulse repetition frequency (i.e., the number of pulses each second), S represents the number of planes, and N_{b} represents the number of transmit beams per plane. EQUATION 1 is a function of the number B-mode planes because a different focused narrower beam is transmitted for each B-mode plane. EQUATION 1 is not a function of the number C-mode slices at least since multiple C-mode slices can be acquired from the same volume of data acquired with the wider beam transmission.

The console 806 further includes a receive circuit 818 configured to receive analog electrical signals for the plane acquisition mode 902, the slice acquisition mode 904, and the plane+slice acquisition mode 906. In one instance, the receive circuit 818 is further configured to pre-process the analog electrical signals, e.g., amplify, digitize, focus, and/or otherwise process the analog electrical signals. For example, in one instance the receive circuit 818 includes an amplifier and a corresponding analog to digital converter (ADC) for each element, where each amplifier amplifies a corresponding analog electrical signal from a micro-volt level to a voltage range of the ADC.

The console 806 further includes a switch 820 configured to switch between the transmit circuit 814 and the receive circuit 818, e.g., by electrically connecting the transmit circuit 814 to the transducer array 810 for a transmit operation and electrically connecting the receive circuit 818 to the transducer array 810 for a receive operation. In an alternative instance, separate switches are employed for each of the transmit circuit 814 and the receive circuit 818.

The console 806 further includes a beamformer 822. For receive operations, the beamformer 822 is configured to beamform, e.g., via delay-and-sum (e.g., a matched-filter beamformer, etc.) and/or other beamforming, the signals from the receive circuit 818 and construct radiofrequency (RF) data for the echoes for each receive operation. With delay-and-sum beamforming, the digital signal for each element is delayed to align the signals in time, amplified, and then summed.

Briefly turning to FIG. 10, in one instance, the beamformer 822 includes a hardware beamformer 1002 (“HARDWARE”) and a software beamformer 1004 (“SOFTWARE”). In one instance, the hardware beamformer 1002 beamforms the signals acquired for generating B-mode planes, and the software beamformer 1004 beamforms the signals acquired for generating C-mode slices. In general, the hardware beamformer 1002 beamforms sequential samples received for a particular B-mode plane to display, whereas the software beamformer 1004 beamforms a subset of the samples within the acquired volume of data that correspond to the C-mode slice with the volume of data to display.

Briefly turning to FIG. 11, in one instance, the hardware beamformer 1002 includes J beamformers, including a beamformer 1102₁, . . . , and 1102_{J}, where J is a positive integer equal to or greater than one. Examples of J include two (2), four (4), eight (8), sixteen (16), thirty-two (32), sixty-four (64), etc. In this instance, the hardware beamformer 1002 can beamform multiple lines in parallel with each transmission. For example, in one instance, the hardware beamformer 1002 includes four (4) beamformers (i.e., J=4) and beamforms four lines each transmission. Returning to FIG. 10, the software beamformer 1004 can form all samples of a C-mode slice each transmission.

Returning to FIG. 8, the console 806 further includes an image generator 824. The image generator 824 processes the beamformed data and constructs, for B-mode planes, one or more B-mode planes of scanlines, and, for C-mode slices, samples on a grid (e.g., pixel-based beamforming) for one or more C-mode slices. In general, the image generator 824 is configured to perform processing such as filtering (e.g., via a Finite Impulse Response (FIR) filter, an Infinite Impulse Response (IIR) filter, etc.), time gain compensation (TGC), I/Q demodulation, envelope detection, logarithmic compression, noise rejection, and/or other processing. When configured for I/Q demodulation, the image generator 824 down mixes the RF signal and, optionally, apply low pass filtering and/or decimation. This may include employing a Hilbert Transform, a combination of a Complex-Demodulation Band Pass Filter and optional decimation, and/or other processing.

The image generator 824 detects and extracts the envelope (e.g., an amplitude) of the I/Q signal (where the image generator 824 I/Q demodulates the RF signal) or the RF signal (when the image generator 824 does not I/Q demodulate the RF signal). In one instance, this is achieved using a Hilbert transform and/or other approach. The image generator 824 compresses the extracted envelope, reducing the dynamic range thereof, e.g., to reduce the dynamic range to a predetermined display precision by a logarithmic (log)-based dynamic range compression and/or otherwise, and outputs a scanline. For B-mode images, the image generator 824 outputs the processed scanlines as a frame/image (i.e., a B-mode image). For C-mode images, in one instance, the image generator 824 outputs the grid as a frame/image (i.e., a C-mode image).

The console 806 further includes a scan converter 826 and a display 828. The scan converter 826 is configured to scan convert the B-mode plane images and the C-mode slice images into a coordinate system for the display 828. The scan converter 826 can be configured to employ analog and/or digital scan converting techniques. In one instance, the display 828 is integrated with the console 806. In another instance, the display 828 is a separate and/or remote display monitor in electrical communication with the console 806. The display 828 allows for displaying one or more images, such as B-mode plane images, C-mode slice images, etc.

Briefly turning to FIG. 12, an example display of B-mode planes and C-mode slices via the display 828 is schematically illustrated. In this example, a user has selected a display option that includes R B-mode planes, including a B-mode plane 1202₁, . . . , and a B-mode plane 1202_{R}, where R is a positive integer equal to or greater than one, and S C-mode slices, including a C-mode slice 1204₁, . . . , and a C-mode slice 1204_{S}, where S is a positive integer equal to or greater than one. For example, in one instance, the display 828 visually presents a first plane in azimuth, a second plane in elevation, and a slice.

In one instance, indicia is visually presented to indicate the location of the slice in connection with the planes. For example, in one instance, indicia is overlayed over the planes such that each plane indicates a location of the other plane and slice relative to the displayed plane. In the illustrated example, the example display further includes controls 1206. The controls allow the user to select one or more B-mode planes and/or one or more C-mode slices to display. In one instance, the controls 1206 can also be utilized to display other information, e.g., a three-dimensional (3-D) view based on the acquired data.

Returning to FIG. 8, the console 806 includes a user interface (U/I) 830. The user interface 830 includes one or more input devices (such as a button, a knob, a slider, a touch screen, a mouse, a keyboard, etc.) and/or other input device, and/or one or more output devices such as a visible, audible, etc. indicator. The user interface 830 is shown integrated with the console 806. In another instance, the user interface 830 is a separate and/or remote keyboard, keypad, touch screen, etc. in electrical communication with the console 806. The user interface 830 allows a user to control an operation of the ultrasound imaging system 802. For example, the user interface 830 allows a user to select a scan protocol and/or enter/select scan parameters. In one instance, the scan protocol and/or parameters identify a type of scanning mode.

The console 806 includes a controller 832. The controller 832 includes a processor(s) such as a microprocessor (μP), a central processing unit (CPU), a graphics processing unit (GPU), etc., and memory, which stores the adaptive spatial compounding algorithm described herein. The controller 832 is configured to control one or more of the transmit circuit 814, the receive circuit 818, the switch 820, the beamformer 822, the image generator 824, the scan converter 826, the display 828, and the user interface 830. One or more of the components of the console 806 can be implemented in software and/or hardware.

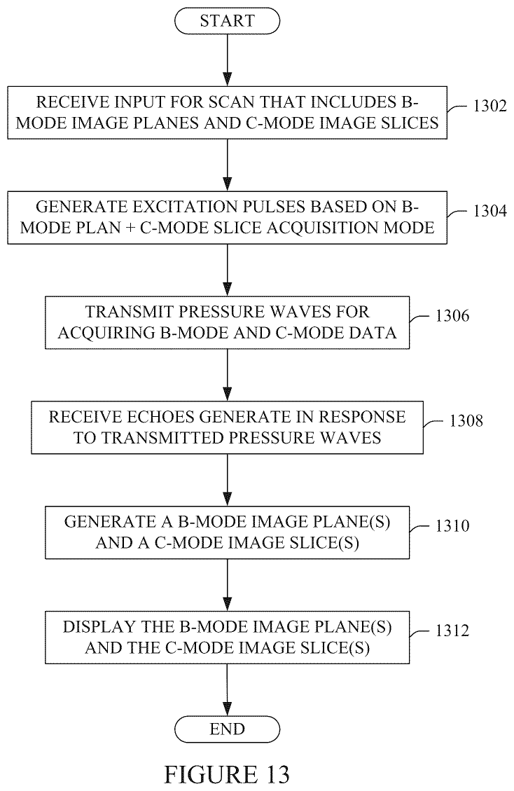

FIG. 13 illustrates a non-limiting example of a flow chart for a computer-implemented method for generating a B-mode image plane(s) and a C-mode image slice(s) for a combined B-mode image plane/C-mode image slice scan. It is to be appreciated that the ordering of the acts in the method is not limiting. As such, other orderings are contemplated herein. In addition, one or more acts may be omitted, and/or one or more additional acts may be included.

At 1302, the user interface 830 receives a user input selecting a scan that includes B-mode image planes and C-mode image slices, as described herein and/or otherwise. At 1304, the controller 832 controls the transmit circuit 814 to generate excitation pulses based on the plane+slice acquisition mode 906 of the acquisition modes 816, as described herein and/or otherwise. At 1306, the 2-D matrix transducer array 810 transmits pressures waves for acquiring data for generating one or more B-mode image planes and generates pressure waves for acquiring a volume of data for generating one or more C-mode image slices, as described herein and/or otherwise.

At 1308, the 2-D matrix transducer array 810 receives echoes corresponding to the transmitted pressure waves for the B-mode image plane acquisition and the C-mode image slice acquisition, as described herein and/or otherwise. At 1310, the echoes for the B-mode image plane acquisition and the C-mode image slice acquisition are processed to generate at least one B-mode image plane and at least one C-mode image slice, as described herein and/or otherwise. At 1312, the at least one B-mode image plane and the at least one C-mode image slice are displayed via the user interface 830, as described herein and/or otherwise.

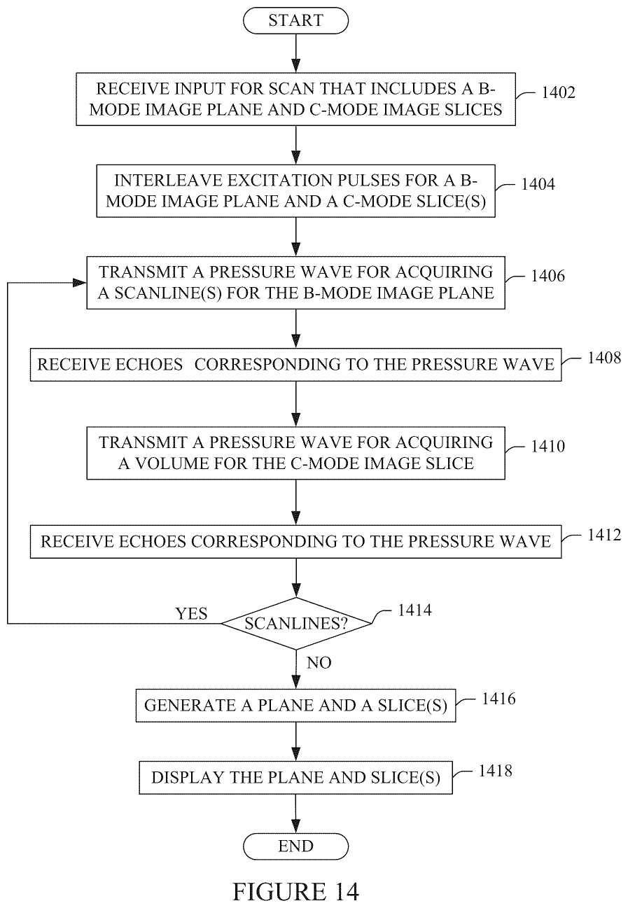

FIG. 14 illustrates a non-limiting example of a flow chart for a computer-implemented method for generating a B-mode image plane and a C-mode image slice(s) for a combined B-mode image plane/C-mode image slice scan. It is to be appreciated that the ordering of the acts in the method is not limiting. As such, other orderings are contemplated herein. In addition, one or more acts may be omitted, and/or one or more additional acts may be included.

At 1402, the user interface 830 receives a user input selecting a scan that includes B-mode image planes and C-mode image slices, as described herein and/or otherwise. At 1404, the controller 832 controls the transmit circuit 814 to interleave excitation pulses for generating a B-mode image plane and one or more C-mode slices, as described herein and/or otherwise. At 1406, the 2-D matrix transducer array 810 transmits pressures waves for acquiring scanlines for generating the B-mode image plane, as described herein and/or otherwise. At 1408, the 2-D matrix transducer array 810 receives echoes corresponding to the transmitted pressure waves for the scanlines for B-mode image plane acquisition, as described herein and/or otherwise. At 1410, the 2-D matrix transducer array 810 transmits pressures waves for acquiring a volume of data for generating the one or more C-mode image slices, as described herein and/or otherwise.

At 1412, the 2-D matrix transducer array 810 receives echoes corresponding to the transmitted pressure waves for the volume of data for generating the one or more C-mode image slices, as described herein and/or otherwise. In some instances, the one or more C-mode image slices are displayed. At 1414, it is determined whether more scanlines are required to generate the B-mode image plane. If it is determined that more scanlines are required to generate a scanplane, acts 1406-1414 are repeated. If a set of scanlines for generating a scanplane are acquired, at 1412, the B-mode image plane and the C-mode image slice(s) are generated, as described herein and/or otherwise. At 1414, the at least one B-mode image plane and C-mode image slice are(s) displayed via the display 828, as described herein and/or otherwise.

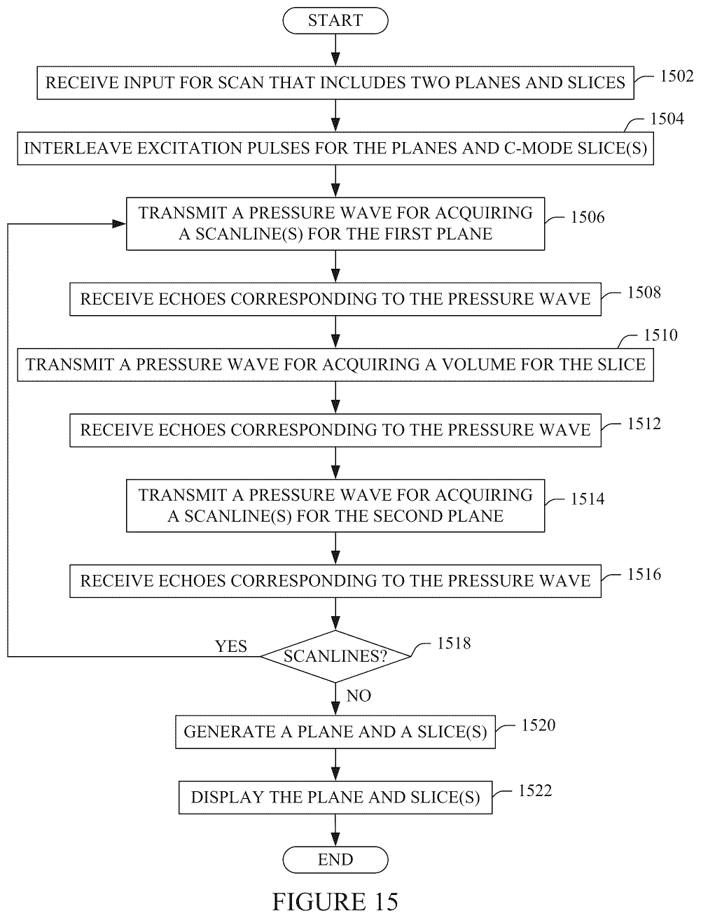

FIG. 15 illustrates a non-limiting example of a flow chart for a computer-implemented method for generating two B-mode image planes and a C-mode image slice(s) for a combined B-mode image plane/C-mode image slice scan. It is to be appreciated that the ordering of the acts in the method is not limiting. As such, other orderings are contemplated herein. In addition, one or more acts may be omitted, and/or one or more additional acts may be included.

At 1502, the user interface 830 receives a user input selecting a scan that includes B-mode image planes and C-mode image slices, as described herein and/or otherwise. At 1504, the controller 832 controls the transmit circuit 814 to interleave excitation pulses for generating two B-mode image planes and one or more C-mode slices, as described herein and/or otherwise. At 1506, the 2-D matrix transducer array 810 transmits pressures waves for acquiring scanlines for generating a first B-mode image plane, as described herein and/or otherwise. At 1508, the 2-D matrix transducer array 810 receives echoes corresponding to the transmitted pressure waves for the scanlines for the first B-mode image plane acquisition, as described herein and/or otherwise.

At 1510, the 2-D matrix transducer array 810 transmits pressures waves for acquiring a volume of data for generating the one or more C-mode image slices, as described herein and/or otherwise. At 1512, the 2-D matrix transducer array 810 receives echoes corresponding to the transmitted pressure waves for the volume of data for generating the one or more C-mode image slices, as described herein and/or otherwise. In some instances, the one or more C-mode image slices are displayed. At 1514, the 2-D matrix transducer array 810 transmits pressures waves for acquiring scanlines for generating a second B-mode image plane, as described herein and/or otherwise. At 1516, the 2-D matrix transducer array 810 receives echoes corresponding to the transmitted pressure waves for the scanlines for the second B-mode image plane acquisition, as described herein and/or otherwise.

At 1518, it is determined whether more scanlines are required to generate the first and the second B-mode image planes. If it is determined that more scanlines are required to generate entire scanplane for the first and the second B-mode image planes, acts 1506-1518 are repeated. If sets of scanlines for generating scanplanes for the first and the second B-mode image planes are acquired, at 1520, the two B-mode image planes and the C-mode image slice(s) are generated, as described herein and/or otherwise. At 1522, the two B-mode image planes and the C-mode image slice(s) are displayed via the display 828, as described herein and/or otherwise.

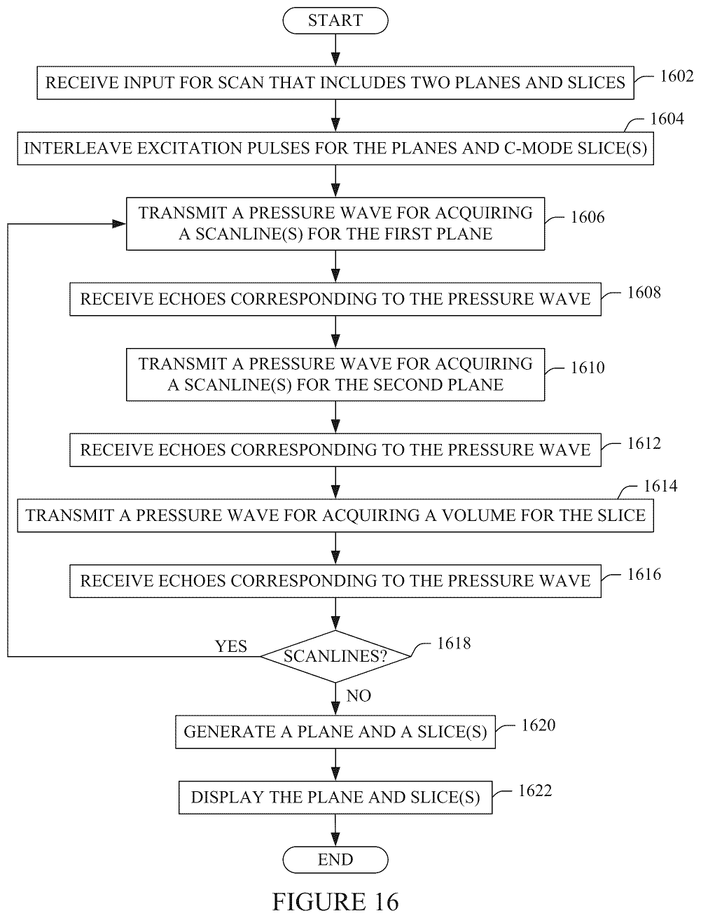

FIG. 16 illustrates a non-limiting example of a flow chart for a computer-implemented method for generating two B-mode image planes and a C-mode image slice(s) for a combined B-mode image plane/C-mode image slice scan. It is to be appreciated that the ordering of the acts in the method is not limiting. As such, other orderings are contemplated herein. In addition, one or more acts may be omitted, and/or one or more additional acts may be included.

At 1602, the user interface 830 receives a user input selecting a scan that includes B-mode image planes and C-mode image slices, as described herein and/or otherwise. At 1604, the controller 832 controls the transmit circuit 814 to interleave excitation pulses for generating two B-mode image planes and one or more C-mode slices, as described herein and/or otherwise. At 1606, the 2-D matrix transducer array 810 transmits pressures waves for acquiring scanlines for generating a first B-mode image plane, as described herein and/or otherwise. At 1608, the 2-D matrix transducer array 810 receives echoes corresponding to the transmitted pressure waves for the scanlines for the first B-mode image plane acquisition, as described herein and/or otherwise.

At 1610, the 2-D matrix transducer array 810 transmits pressures waves for acquiring scanlines for generating a second B-mode image plane, as described herein and/or otherwise. At 1612, the 2-D matrix transducer array 810 receives echoes corresponding to the transmitted pressure waves for the scanlines for the second B-mode image plane acquisition, as described herein and/or otherwise. At 1614, the 2-D matrix transducer array 810 transmits pressures waves for acquiring a volume of data for generating the one or more C-mode image slices, as described herein and/or otherwise. At 1616, the 2-D matrix transducer array 810 receives echoes corresponding to the transmitted pressure waves for the volume of data for generating the one or more C-mode image slices, as described herein and/or otherwise. In some instances, the one or more C-mode image slices are displayed.

At 1618, it is determined whether more scanlines are required to generate the first and the second B-mode image planes. If it is determined that more scanlines are required to generate entire scanplane for the first and the second B-mode image planes, acts 1606-1618 are repeated. If sets of scanlines for generating scanplanes for the first and the second B-mode image planes are acquired, at 1620, the two B-mode image planes and the C-mode image slice(s) are generated, as described herein and/or otherwise. At 1622, the two B-mode image planes and the C-mode image slice(s) are displayed via the display 828, as described herein and/or otherwise.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only and are thus not intended to limit in any way the definition and/or meaning of the term “computer.” The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used in practice as some jurisdictions require them, but this should not be taken to mean that the features in the dependent claims are mutually exclusive.

1. An ultrasound imaging system, comprising:
a transmit circuit configured to generate ultrasound element excitation pulses based on an acquisition mode that includes at least two different types of ultrasound acquisitions for a single ultrasound imaging scan, wherein the at least two different types include focused narrower transmission beams for B-mode planes and wider transmission beams for C-mode slices;
a transducer array with elements configured to transmit ultrasound pressure waves based on the at least two different types of ultrasound acquisitions, including interleaving the focused narrower transmission beams to acquire scanplanes of data for the B-mode planes and the wider transmission beams to acquire the volume data for the C-mode slices and receive, for each of the at least two different types of ultrasound acquisitions, a corresponding echo signal;
a beamformer configured to independently beamform the echo signals for the at least two different types of ultrasound acquisitions;
an image generator configured to generate an image for each of the at least two different types of ultrasound acquisitions based on the corresponding echo signal; and
a display configured to concurrently display the images for each of the at least two different types of ultrasound acquisitions.

2. The ultrasound imaging system of claim 1, wherein one of the at least two different types of ultrasound acquisitions includes an image plane acquisition and another of the at least two different types of ultrasound acquisitions includes an image slice acquisition.

3. The ultrasound imaging system of claim 2, wherein the transducer array interleaves transmissions for the image plane and the image slice.

4. The ultrasound imaging system of claim 3, wherein the transducer array transmits a first pressure wave for acquiring a sub-set of scanlines for the image plane and a second pressure wave for acquiring a volume of data for the image slice.

5. The ultrasound imaging system of claim 2, wherein the transducer array interleaves transmissions for two image planes and the image slice.

6. The ultrasound imaging system of claim 5, wherein the transducer array transmits a first pressure wave for acquiring a sub-set of scanlines for a first image plane, a second pressure wave for acquiring a volume of data for the image slice, and a third pressure wave for acquiring a sub-set of scanlines for a second image plane.

7. The ultrasound imaging system of claim 6, wherein the first image plane is in azimuth and the second image plane is in elevation.

8. The ultrasound imaging system of claim 5, wherein the transducer array transmits a first pressure wave for acquiring a sub-set of scanlines for a first image plane, a second pressure wave for acquiring a sub-set of scanlines for a second image plane, and a third pressure wave for acquiring a volume of data for the image slice.

9. The ultrasound imaging system of claim 8, wherein the first image plane is in azimuth and the second image plane is in elevation.

10. The ultrasound imaging system of claim 1, wherein the image includes a first image plane, and the first image plane has a first image quality that is greater than a second image quality of a second image generated from a volume of data, given a same frame rate.

11. A method, comprising:
generating ultrasound element excitation pulses based on an acquisition mode that includes at least two different types of ultrasound acquisitions for a single ultrasound imaging scan, wherein the at least two different types include focused narrower transmission beams for B-mode planes and wider transmission beams for C-mode slices;
transmitting ultrasound pressure waves based on the at least two different types of ultrasound acquisitions, including interleaving the focused narrower transmission beams to acquire scanplanes of data for the B-mode planes and the wider transmission beams to acquire the volume data for the C-mode slices;
receiving, for each of the at least two different types of ultrasound acquisitions, a corresponding echo signal;
beamforming the echo signals for the at least two different types of ultrasound acquisitions;
generating an image for each of the at least two different types of ultrasound acquisitions based on the corresponding echo signal; and
displaying, concurrently, the images for each of the at least two different types of ultrasound acquisitions.

12. The method of claim 11, wherein one of the at least two different types of ultrasound acquisitions includes an image plane acquisition and another of the at least two different types of ultrasound acquisitions includes an image slice acquisition, and further comprising:
interleaving transmissions for the image plane and the image slice.

13. The method of claim 12, wherein the interleaving
comprises:
transmitting a first pressure wave for acquiring a first sub-set of scanlines for the image plane;
receiving a first echo signal in response to the first pressure wave;
transmitting a second pressure wave for acquiring a volume of data for the image slice;
receiving a second echo signal in response to the second pressure wave;
transmitting at least a third pressure wave for acquiring a second sub-set of scanlines for the image plane;
receiving at least a third echo signal in response to the third pressure wave; and
generating the image plane based on the first and third echo signals and the image slice based on the second echo signal.

14. The method of claim 13, wherein the interleaving
comprises:
transmitting a first pressure wave for acquiring a first sub-set of scanlines for a first image plane;
receiving a first echo signal in response to the first pressure wave;
transmitting a second pressure wave for acquiring a volume of data for the image slice;
receiving a second echo signal in response to the second pressure wave;
transmitting a third pressure wave for acquiring a first sub-set of scanlines for a second image plane;
receiving a third echo signal in response to the third pressure wave; and
generating the first image plane based on the first echo signal, the image slice based on the second echo signal, and the second image plane based on the third echo signal.

15. The method of claim 13, wherein the interleaving
comprises:
transmitting a first pressure wave for acquiring a first sub-set of scanlines for a second image plane;
receiving a first echo signal in response to the first pressure wave;
transmitting a second pressure wave for acquiring a first sub-set of scanlines for a second image plane;
receiving a second echo signal in response to the second pressure wave;
transmitting a third pressure wave for acquiring a volume of data for the image slice;
receiving a third echo signal in response to the third pressure wave; and
generating the first image plane based on the first echo signal, the second image plane based on the second echo signal, and the image slice based on the third echo signal.

16. A non-transitory computer readable medium encoded with computer executable instructions, which, when executed by a processor, cause the processor to:
generate ultrasound element excitation pulses based on an acquisition mode that includes at least two different types of ultrasound acquisitions for a single ultrasound imaging scan, including a B-mode plane and a C-mode slice;
transmit ultrasound pressure waves based on the at least two different types of ultrasound acquisitions by interleaving a focused narrow beam transmission to acquire scanlines for the B-mode plane and a wide beam transmission to acquire a volume of data for the C-mode slice;
receive, for each of the at least two different types of ultrasound acquisitions, a corresponding echo signal;
beamform the echo signals for the at least two different types of ultrasound acquisitions;
generate an image for each of the at least two different types of ultrasound acquisitions based on the corresponding echo signal; and
display, concurrently, the images for each of the at least two different types of ultrasound acquisitions.

17. The non-transitory computer readable medium of claim 16, wherein one of the at least two different types of ultrasound acquisitions includes an image plane acquisition and another of the at least two different types of ultrasound acquisitions includes an image slice acquisition, and the computer executable instructions further cause the processor to:
interleave transmissions for the image plane and the image slice.

18. The non-transitory computer readable medium of claim 17,
wherein the computer executable instructions further cause the processor to:
transmit a first pressure wave for acquiring a first sub-set of scanlines for the image plane;
receive a first echo signal in response to the first pressure wave;
transmit a second pressure wave for acquiring a volume of data for the image slice;
receive a second echo signal in response to the second pressure wave;
transmit at least a third pressure wave for acquiring a second sub-set of scanlines for the image plane;
receive at least a third echo signal in response to the third pressure wave; and
generate the image plane based on the first and third echo signals and the image slice based on the second echo signal.

19. The non-transitory computer readable medium of claim 16,
wherein the computer executable instructions further cause the processor to:
transmit a first pressure wave for acquiring a first sub-set of scanlines for a first image plane;
receive a first echo signal in response to the first pressure wave;
transmit a second pressure wave for acquiring a volume of data for the image slice;
receive a second echo signal in response to the second pressure wave;
transmit a third pressure wave for acquiring a first sub-set of scanlines for a second image plane;
receive a third echo signal in response to the third pressure wave; and
generate the first image plane based on the first echo signal, the image slice based on the second echo signal, and the second image plane based on the third echo signal.

20. The non-transitory computer readable medium of claim 17,
wherein the computer executable instructions further cause the processor to:
transmit a first pressure wave for acquiring a first sub-set of scanlines for a second image plane;
receive a first echo signal in response to the first pressure wave;
transmit a second pressure wave for acquiring a first sub-set of scanlines for a second image plane;
receive a second echo signal in response to the second pressure wave;
transmit a third pressure wave for acquiring a volume of data for the image slice;
receive a third echo signal in response to the third pressure wave; and
generate the first image plane based on the first echo signal, the second image plane based on the second echo signal, and the image slice based on the third echo signal.
